(19) United States
(12) Patent Application Publication
(10) Pub. No.: US 2009/0182196 A1
(43) Pub. Date: Jul. 16, 2009

(54) METHOD AND APPARATUS FOR MANUALLY GUIDING AN ENDOSCOPE

(75) Inventor: John Kefer, Cleveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: THE CLEVELAND CLINIC FOUNDATION

(21) Appl. No.: 12/261,379

(22) Filed: Oct. 30, 2008

Related U.S. Application Data
(60) Provisional application No. 60/983,610, filed on Oct. 30, 2007.

(51) Int. Cl. A61B 1/01

(52) U.S. Cl. 600/114

(57) ABSTRACT

An endoscopic guidance system is provided. The system comprises an endoscope and a first rail assembly having a rail member and a first rod secured thereto. The first rod is rotatable relative to the rail member by rotation means. A sleeve assembly is also provided. The sleeve assembly is coupled to the first rod, wherein the endoscope is releasably engageable with the sleeve assembly. Rotation of the first rod causes the sleeve assembly to move along the first rod to guide the endoscope to a desired position along a first degree of freedom.

### RELATED APPLICATION

This application claims priority from U.S. Provisional Application No. 60/983,610, filed Oct. 30, 2007, the subject matter of which is incorporated herein by reference.

### TECHNICAL FIELD

The present invention is directed to an endoscopic guidance system, and in particular, is directed to a telesurgical endoscopic guidance system that utilizes a number of rails and control knobs to allow a practitioner to precisely guide the endoscope along several degrees of freedom.

### BACKGROUND OF THE INVENTION

Current surgical procedures, such as flexible cystoscopy, uteteroscopy, and other endoscopic diagnostic and therapeutic procedures in other organ systems require at least two skilled surgeons to be actively involved during the case. One surgeon drives the endoscope, ensuring that the scope is positioned properly at all times, and the other surgeon threads endoscopic tools down the lumen of the endoscope to perform the diagnostic or therapeutic procedure through the end of the endoscope. During these procedures, both surgeons are constantly busy—one maintaining adequate orientation and exposure to the treatment area, and the other constantly manipulating the small endoscopic tools to perform the procedure.

The present invention allows one surgeon to perform both tasks. The surgeon can guide the scope into place, mount the endoscope into the guidance system 10 for absolute positional stability, and then proceed confidently with the treatment phase of manipulating small tools within the lumen of the endoscope. By freeing one surgeon from the procedure, the therapeutic and financial efficiency for the entire surgical practice substantially increases.

The present invention accomplishes this by utilizing a hand-guided, motorless system to guide the endoscope. The system enables high-precision calibration in many degrees of freedom to ensure accurate orientation of the endoscope. Color-coded dials controlling each degree of freedom of movement allow for the potential for telesurgically guided endoscopy, where a remote endoscopist provides direction to a bedside assistant to direct endoscopic maneuvers (i.e. Endoscopist to the Assistant—“ . . . advance the scope 2 cm using the red dial . . . ”).

The present invention is also designed to function with any endoscope, and so can be used by all surgical specialties that perform diagnostic and therapeutic endoscopy, including general surgery, otolaryngology (ENT), colorectal surgery, and gastroenterology.

### SUMMARY OF THE INVENTION

In accordance with the present invention, an endoscopic guidance system is provided. The system comprises an endoscope and a first rail assembly having a rail member and a first rod secured thereto. The first rod is rotatable relative to the rail member by rotation means. A sleeve assembly is also provided. The sleeve assembly is coupled to the first rod, wherein the endoscope is releasably engageable with the sleeve assembly. Rotation of the first rod causes the sleeve assembly to move along the first rod to guide the endoscope to a desired position along a first degree of freedom.

In accordance with another exemplary embodiment of the present invention, a method is provided. The method comprises the steps of providing an endoscope and a first rail assembly having a rail member and a first rod secured thereto, the first rod being rotatable relative to the rail member by rotation means. The method further comprises the steps of providing a sleeve assembly coupling the sleeve assembly to the first rod. The endoscope is then secured within the sleeve assembly. The first rod is rotated to cause the sleeve assembly to move along the first rod to guide the endoscope to a desired position along a first degree of freedom.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a schematic illustration of a guidance system in accordance with the present invention;

FIG. 2 is an isometric view of a first rail assembly;

FIG. 3 is a top view of the base of a carriage assembly;

FIG. 4 is a sectional view of the base of FIG. 3 taken along line 4-4;

FIG. 5 is a top view of the carriage assembly;

FIG. 6A is a sectional view of the carriage assembly of FIG. 5 taken along line 6A-6A;

FIG. 6B is a sectional view of the carriage assembly of FIG. 5 taken along line 6B-6B;

FIG. 7A is an exploded view of the second rail assembly and sleeve assembly;

FIG. 7B is an isometric view of the second rail assembly and sleeve assembly in the assembled condition;

FIG. 8 is a side view of the carriage assembly coupled to the second rail assembly;

FIG. 9 is a side view of the guidance system to a table;

FIG. 10 is a schematic illustration of the guidance system in the assembled condition;

FIG. 11 is a schematic illustration of a guidance system in accordance a further embodiment of the present invention;

FIG. 12A is an enlarged view of an articulating stage of FIG. 11;

FIG. 12 B is a side view of the stage of FIG. 12A;

FIG. 13 is an enlarged view of the stage of FIG. 12A in a first condition;

FIG. 14 is an enlarged view of the stage of FIG. 12A in a second condition;

FIG. 15 is a section view of the sheath of FIG. 11 taken along line 15-15; and

FIG. 16 is an enlarged view of the wheel and toothed cylinder of the sheath of FIG. 15.

### DETAILED DESCRIPTION

The present invention is directed to an endoscopic guidance system, and in particular, is directed to a telesurgical endoscopic guidance system that utilizes a number of rails and control knobs to allow a practitioner to precisely guide the endoscope along several degrees of freedom.

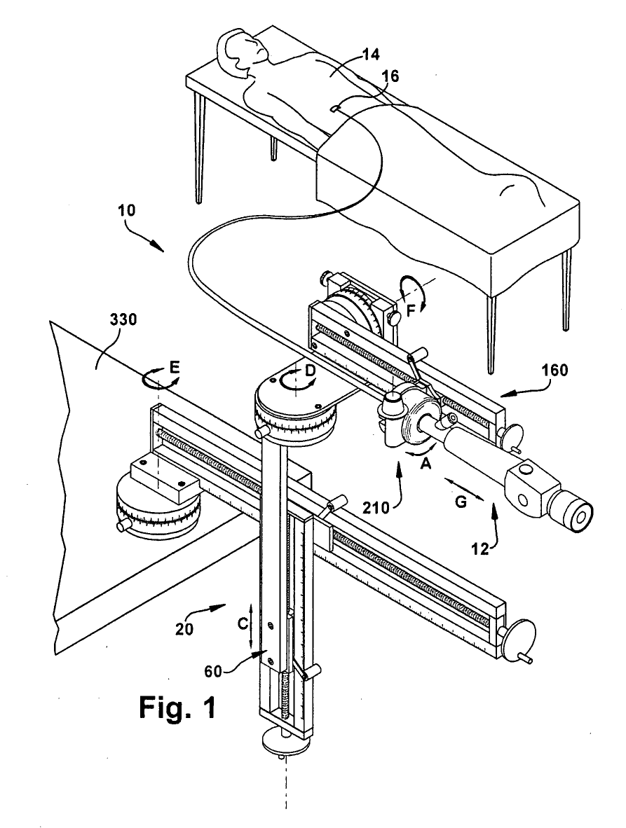

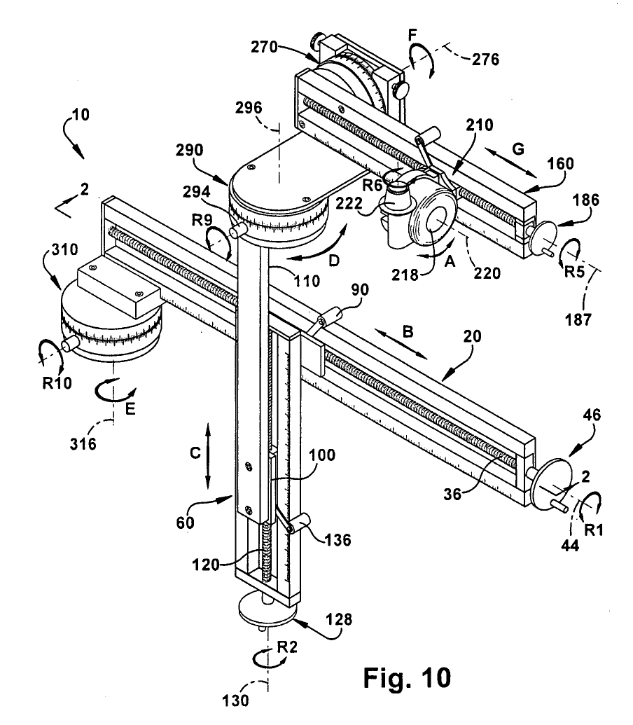

A guidance system 10 in accordance with a first embodiment of the present invention is depicted schematically in FIG. 1. The system comprises a first rail assembly 20, a second rail assembly 160, a carriage assembly 60, and a sleeve assembly 210. The system 10 is mounted to a table 330 for stability, and is used for precision movement and articulation of an endoscope 12 disposed within the sleeve assembly 210. The endoscope 12 is used to access an incision 16 or body cavity of a patient 14. The system 10 provides for multiple degrees of freedom of motion of the endoscope 12. These ranges of motion are illustrated generally by letters A-G in FIGS. 1 and 10. This system can be constructed of metal, plastic or any other light-weight durable material.

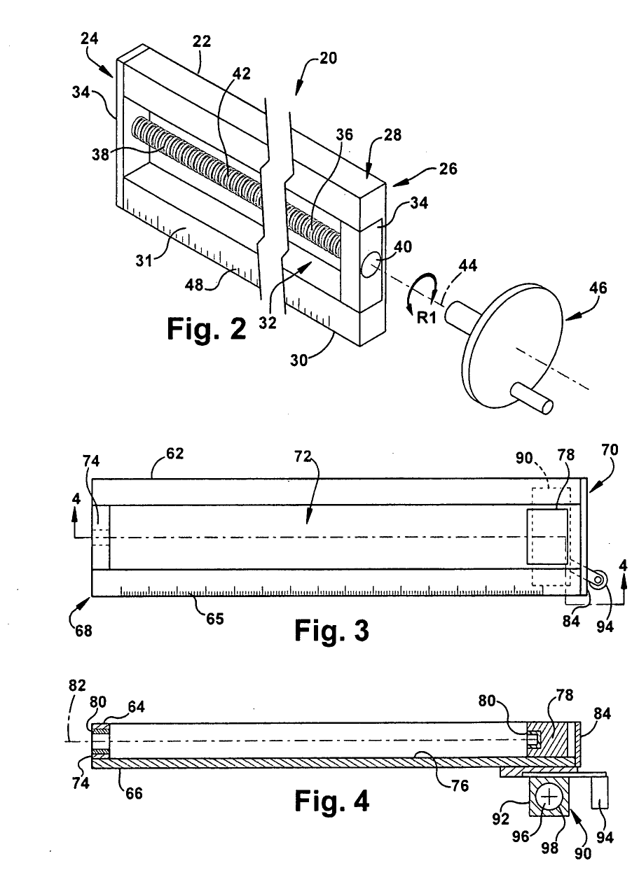

FIG. 2 depicts the first rail assembly 20. The rail assembly 20 comprises a rail 22 having a generally C-shaped cross section, although alternative cross-sections can be used. The rail 22 has a top surface 28 and a substantially parallel bottom surface 30 that each extend from a proximal end 24 to a distal end 26. A passage 32 extends between the top surface 28 and the bottom surface 30 and from the proximal end 24 to the distal end 26 of the rail 22. A front surface 31 of the rail 22 includes measuring indicia 48 from the proximal end 24 to the distal end 26, the purpose of which will be hereafter described. The rail 22 is made of steel, aluminum, or any other comparable light-weight, yet durable, material.

A bearing or rotation means 34 is secured at both the proximal end 24 and the distal end 26 of the rail 22 within the passage 32. The bearings 34 may be typical roller bearings having ball bearings or the like. A rod 36 is disposed within the passage 32 and includes external threads 42 substantially along its length. The external threads 42 may be helical, square, or the like. The rod 36 includes ends 38, 40 that are smooth and sized to be press fit within each bearing 34 on the rail 22. The press fit between each end 38, 40 of the rod 36 and each bearing 34, coupled with each bearing being secured relative to the rail 22, allows the rod 36 to rotate freely about its central axis 44 (indicated by “R1”) within the bearings without any relative movement between the bearings 34 and the rail. This configuration also allows the rod 36 to rotate within the bearings 34 without the rod moving longitudinally relative to either the bearings 34 or the rail 22. A knob or rotation means 46 may be connected to the end 40 of the rod 36 and outside of the rail 22 to facilitate rotation of the rod by the practitioner.

A base 62 that comprises part of the carriage assembly 60 is depicted in FIGS. 3-4. The base 62 is generally rectangular and extends from a proximal end 68 to a distal end 70. The base 62 includes a top surface 64 and a substantially parallel bottom surface 66. The top surface 64 bears measuring indicia 65 from the proximal end 68 to the distal end 70 for purposes that will be hereafter described. The base 62 includes a passage 72 that extends from the top surface 64 to an inner surface 76 that is spaced between the top surface and the bottom surface 66. The inner surface 76 is substantially parallel to the top surface 64 and the bottom surface 66. The passage 72 also extends from the distal end 70 towards the proximal end 68, and terminates at an end wall 74 at the proximal end 68. An end plate 84 is secured to the distal end 70 of the base 62 to close the passage 72 at the distal end. The base 62 is constructed of steel, aluminum, or the like.

A stop 78 having a substantially square or rectangular shape is secured to, or integrally formed with, the inner surface 76 of the base 62, and extends away from the inner surface towards the top surface 64 of the base. Similar to the first rail assembly 20, the stop 78 and the end wall 74 each include a bearing or rotation means 80. The bearings 80 in the stop 78 and the end wall 74, respectively, are aligned along a central axis 82 extending substantially parallel to the top surface 64 and the bottom surface 66 of the base 62. The central axis 82 also extends through the midline of the end wall 74 and the end plate 84.

A first locking mechanism 90 is secured to the bottom surface 66 at the distal end 70 of the base 62. The first locking mechanism 90 includes a clamp portion 92 having an opening 96 and a lever 94 that extends from the clamp portion, past the end plate 84, and then away from the bottom surface of the base 62. This configuration forms an L-shaped lever 94. The opening 96 includes internal threads 98 that are configured to mate with the external threads 42 on the rod 36 in the first rail assembly 20. The threads 98 can be helical, square, or the like so long as the threads 98 can mate with the threads 42. The opening 96 of the first locking mechanism 90 is adapted to expand when the lever 94 is in a first, open condition and contract when the lever is in a second, locked condition. Thus, the locking mechanism 90 is capable of providing an inwardly directed force in the closed condition. As will be hereafter described, the lever 94 operates to selectively engage the clamp 92 with the rod 36 disposed within the passage 32 of the rail 22 in the first rail assembly 20.

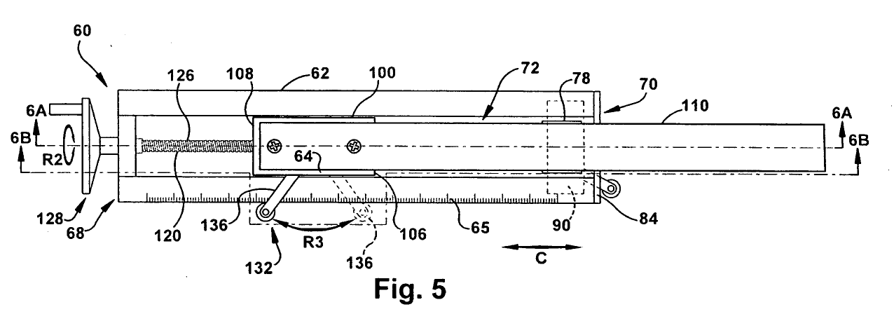

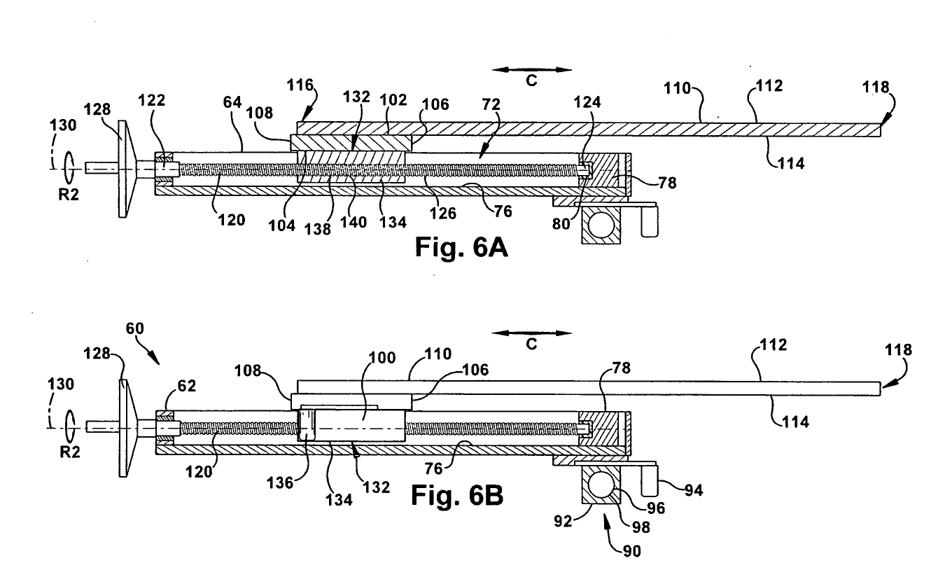

The carriage assembly 60 cooperates with the base 62 and is depicted in the assembled condition in FIGS. 5-6B. The carriage assembly 60 includes a carriage 100 disposed within the passage 72 of the base 62. The carriage 100 is substantially square or rectangular and is constructed of a rigid material, such as steel or aluminum. The carriage 100 includes a top surface 102, a bottom surface 104, a leading edge 106, and a trailing edge 108.

A second locking mechanism 132 is secured to the bottom surface 104 of the carriage 100. The second locking mechanism 132 includes a clamp portion 134 having an opening 138 and a lever 136. The lever 136 extends from the clamp portion 134, beyond the top surface 102 of the carriage 100, and then in a direction away from the carriage and parallel to the top surface, thereby forming an L-shape. The opening 138 includes internal threads 140 that are configured to mate with a rod 120 having external threads 126. The threads 126, 140 may be square, helical, or the like. The opening 138 of the second locking mechanism 132 is adapted to expand when the lever 136 is in a first, open condition and contract when the lever is in a second, locked condition. Thus, the locking mechanism 132 is capable of providing an inwardly directed force in the closed condition. As will be hereafter described, the lever 136 operates to selectively engage the clamp 134 with the rod 120 disposed within the passage 72 of the base 62.

An arm 110 is fastened to, or integrally formed with, the top surface 102 of the carriage 100. The arm 110 is substantially longer than the carriage 100, and includes a top surface 112 and a bottom surface 114 that extend from a proximal end 116 to a distal end 118. The arm 110 is rectangular and made of a rigid material such that the distal end 118 of the arm 110 does not bend relative to the proximal end 116 when the arm 110 is secured to the carriage 100 and the weight of the distal end of the arm is unsupported.

As shown in FIG. 6A, the second locking mechanism 132 mounts the carriage 100, with the arm 110 secured thereto, to the rod 120. Similar to the rod 36 in the first rail assembly 20, the rod 120 is disposed within the passage 72 of the base 62, and at least a portion of the carriage 100 is positioned in the passage when the carriage is mounted to the rod. The rod 120 has external threads 126 substantially along its length and includes a first end 122 that is smooth and press fit into the bearing disposed within the end wall 74, and a second end 124 that is smooth and press fit into the bearing disposed within the stop 78. The press fit between each end 122, 124 of the rod 120 and each bearing 80 allows the rod to rotate freely with the bearings about the central axis 130 relative to the base 62. This configuration also allows the rod 120 to rotate within the bearings 80 without the rod moving longitudinally relative to either the bearings or the base 62. A knob or rotation means 128 may be connected to the first end 122 of the rod 120 and outside of the base 62 to facilitate rotation of the rod by the practitioner.

The carriage 100 is mounted to the rod 120 by sliding the rod through the opening 138 in the second locking mechanism 132 when the lever 136 is in the open condition, i.e., when the opening is in the expanded condition. Once mounted, the carriage 100 can move relative to both the rod 120 and the base 62. When the carriage 100 is mounted on the rod 120, the carriage and the arm 110 extend substantially parallel to the top surface 64, bottom surface 66, and inner surface 76 of the base 62. The carriage 100 is sized such that the top surface 102 of the carriage extends above the top surface 64 of the base 62. The arm 110 extends along the top surface 102 of the carriage 100 and beyond the stop 78 and the end plate 84 of the base 62. The bottom surface 114 of the arm 110 does not contact the stop 78 or the end plate 74.

The engagement between the opening 138 in the second locking mechanism 132 and the rod 120 allows the carriage 100 to move back and forth within the passage 72 of the base 62 in the manner indicated by “C” in FIG. 6A. The range of motion of the carriage 100 is limited to the distance between the end wall 74 and the stop 78 in the base 62. The carriage 100 can move toward the distal end 70 of the base 62 until the second locking mechanism 132 abuts the stop 78, and toward the proximal end 68 of the base until the second locking mechanism abuts the end wall 74. Movement of the carriage 100 is accomplished by applying a force to the carriage substantially parallel to the rod 120 such that the static friction between the opening 138 in the second locking mechanism 132 and the periphery of the rod is overcome to allow the carriage to slide relative to the rod. This construction allows for quick, substantial movement of the carriage 100 and the arm 110 along the rod 120 to a desired location.

The lever 136 is actuatable to selectively secure the carriage 100 to the rod 120. In particular, the lever 136 operates to selectively engage the threads 140 on the opening 138 of the clamp portion 134 of the second locking mechanism 132 with the threads 126 on the rod 120 by selectively expanding and contracting the opening 138 in the clamp portion relative to the rod. When the lever 136 is in a first condition, indicated by solid lines in FIG. 5, the clamp 134 is not fully engaged with the rod 120 and, thus, there exists only static friction between the clamp and the rod. In this condition, the carriage 100 can be moved along the rod 120 by applying force as noted above to overcome the static friction and make substantial movements of the carriage relative to the rod.

The lever 136 can be moved from the first condition to a second condition by rotating the lever (indicated by “R3”) relative to the clamp portion 134. When the lever 136 is in the second condition, indicated by dashed lines 136 in FIG. 5, the threads 140 on the opening 138 of the clamp 134 engage the threads 126 on the rod 120 and apply a force to the periphery of the rod sufficient to rigidly couple the clamp 134 and, thus, the carriage 100 to the rod 120. In this condition, the carriage 100 cannot be moved along the rod 120 by applying force due to the thread engagement and clamping force applied by the clamp 134 to the rod. Rather, the carriage 100 is moved by turning the rod 120 about its central axis 130 via the knob 128, indicated by “R2”. When the rod 120 is rotated, the threaded engagement between the threads 126 on the rod and the threads 140 on the second locking mechanism 132 causes the carriage assembly 60 to move relative to the fixed rod 120. This occurs due to the first end 122 and the second end 124 of the rod 120 being fixed in place in the bearings 80. This relative movement of the carriage 100 along the rod 120 is in small increments, or fine.

The indicia 65 on the top surface 64 of the base 62 bear incremental measurements, e.g. inches, millimeters, etc., and reflect the amount of longitudinal travel of the carriage 100 along the rod 120. Rotation of the knob 128 in a clockwise direction results in the carriage 100 moving toward the stop 78 on the base 62, whereas rotation of the knob in a counterclockwise direction results in the carriage moving toward the end wall 78 of the base. The relationship between the amount of rotation of the knob 128 and the correlative longitudinal travel distance of the carriage 100 along the rod 120 is based on the fineness of the threaded engagement between the rod 120 and the opening 138 in the clamp 134. For example, rotation of the knob 128 in the clockwise direction 45° may cause the carriage 100 to move 3 mm towards the stop 78 in the carriage assembly 60. However, if more threads per inch are present on the rod 120 and the opening 138 in the clamp 134, the same 3 mm movement of the carriage 100 could require 180° of clockwise rotation of the knob 128. The threaded engagement, therefore, determines how sensitive the carriage 100 is to rotation of the knob 128.

As noted, the first locking mechanism 90 is secured to the bottom surface 66 of the base 62 (FIG. 6B). The carriage assembly 60 is mounted on the first rail assembly 20 by feeding the rod 36 through the opening 96 in the first locking mechanism 90 when the lever 94 is in the open condition, i.e., when the opening 96 is in the expanded condition. This places the carriage assembly 60 substantially orthogonal to the first rail assembly 20 (see FIGS. 1 and 10). Due to the engagement between the opening 96 in the first locking mechanism 90 and the rod 120, the carriage assembly 60 is capable of moving back and forth relative to the passage 32 of the rail 22 in the first rail assembly 20 in the manner indicated by “B” in FIG. 10. The range of travel of the carriage assembly 60 along the rod 36 is limited to the distance between the bearings 34 in the rail 22.

The carriage assembly 60 may move toward the proximal end 24 of the rail 22 of the first rail assembly 20 until the clamp portion 92 of the first locking mechanism 90 abuts the bearing 34 at the proximal end 24 of the rail, and toward the distal end 26 of the rail until the clamp portion 92 abuts the bearing at the distal end 26 of the rail. Movement of the carriage assembly 60 is accomplished by applying a force to the carriage assembly substantially parallel to the rod 36 such that the static friction between the opening 96 in the first locking mechanism 90 and the periphery of the rod is overcome to allow the carriage assembly 60 to slide relative to the rod. This construction allows for quick, substantial movement of the carriage assembly 60 along the rod 36 to a desired location.

The lever 94 is actuatable to selectively secure the carriage assembly 60 to the rod 36. In particular, the lever 94 operates to selectively engage the threads 98 on the opening 96 of the first locking mechanism 90 with the threads 42 on the rod 36 by selectively expanding and contracting the opening in the clamp 92 relative to the rod 36. When the lever 94 is in a first condition (not shown) the clamp 92 is not fully engaged with the rod 36 and, thus, there exists only static friction between the clamp and the rod. In this condition, the carriage assembly 60 can be moved along the rod 36 by applying force, as noted above, to overcome the static friction and make substantial movements of the carriage assembly relative to the rod.

The lever 94 can be moved from the first condition to a second condition by rotating the lever, indicated by “R4”, relative to the clamp portion 92. When the lever 94 is in the second condition, the threads 98 on the opening 96 of the clamp 92 engage the threads 42 on the rod 36 and apply a force to the periphery of the rod 36 sufficient to rigidly couple the rod to the clamp 92 and, thus, to the carriage assembly 60. In this condition, the carriage assembly 60 cannot be moved along the rod 36 by applying force due to the thread engagement and clamping force applied by the clamp 92 to the rod 36. Rather, the carriage assembly 60 is moved by turning the rod 36 about its axis 44 via the knob 46, indicated by “R1” at FIG. 10. When the rod 36 is rotated, the threaded engagement between the threads 42 on the rod and the threads 98 on the opening 96 of the clamp 92 causes the carriage assembly 60 to move relative to the fixed rod. This relative movement of the carriage assembly 60 along the rod 36 is in small increments, or fine.

The indicia 48 on the front surface 31 of the rail 22 bear incremental measurements, e.g., inches, millimeters, etc., and reflect the amount of longitudinal travel of the carriage assembly 60 along the rod 36. Rotation of the knob 46 in a clockwise direction results in the carriage assembly 60 moving toward the proximal end 24 of the rail 22 of the first rail assembly 20, and rotation of the knob in a counterclockwise direction results in the carriage assembly moving toward the distal end 26 of the rail. The relationship between the amount of rotation of the knob 46 and the correlative longitudinal travel distance of the carriage assembly 60 along the rod 36 is based on the fineness of the threaded engagement between the rod and the opening 96 in the clamp 92. For example, rotation of the knob 46 in the clockwise direction 45° may cause the carriage assembly 60 to move 3 mm towards the proximal end 24 of the rail 22. However, if more threads per inch are present on the rod 36 and the opening 96 in the clamp 92, the same 3 mm movement of the carriage assembly 60 may require 180° of clockwise rotation of the knob 46. This threaded engagement, therefore, determines how sensitive the carriage assembly 60 is to rotation of the knob 46.

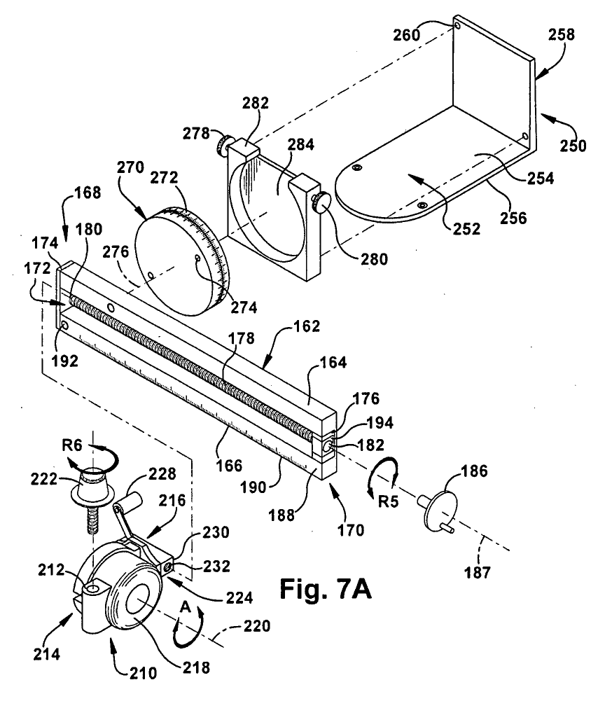

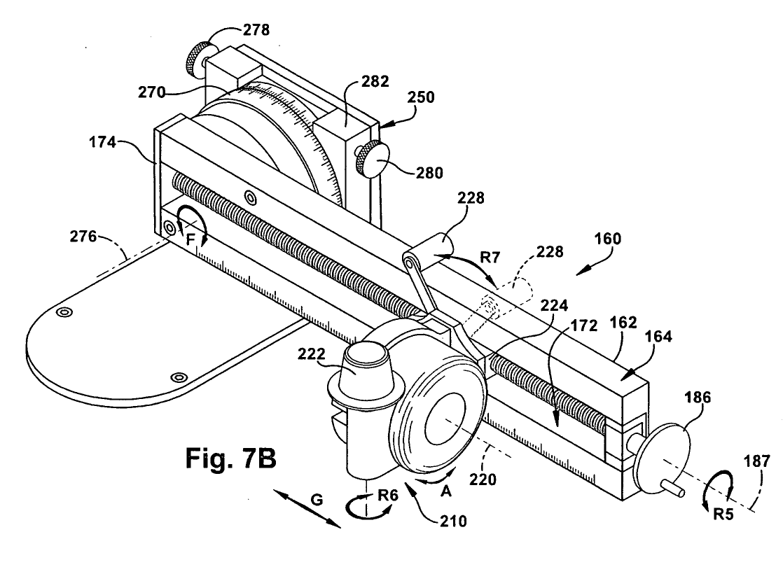

The second rail assembly 160 in accordance with the present invention is depicted in FIGS. 7A-B. The second rail assembly 160 includes a rail 162, a bracket 250, a sleeve assembly 210, and a rotating member 270. The rail 162 has a generally C-shaped cross section having a top surface 164 and a substantially parallel bottom surface 166 that extends from a proximal end 168 to a distal end 170. A passage 172 extends between the top surface 164 and the bottom surface 166 and from the proximal end 168 to the distal end 170. The rail 162 includes a front surface 188 on which measuring indicia 190 are made, the purpose of which will be described hereafter. The rail 162 is made of steel, aluminum, plastic or any other comparable light-weight, yet durable, material.

An end plate 174 is secured to the proximal end 168 of the rail 162 to cover the proximal end and close the passage 172. Another end plate 176 is secured to, and closes, the distal end 170 of the passage 172. A bearing or rotation means 194 is secured at the distal end 170 of the rail 162 within the end plate 176. The bearing 194 may be a typical roller bearing having ball bearings or the like. A rod 178 having a first end 180 and a second end 182 is positioned within the passage 172 and includes external threads 184 substantially along its length. The first end 180 maintains external threads 184 while the second end 182 is smooth and sized to be press fit within the bearing 194 disposed on the end plate 176. The press fit between the second end 182 of the rod 178 and the bearing 194 allows the rod 178 to rotate freely within the bearing 194 about its central axis 187 relative to the rail 162. This configuration also allows the rod 178 to rotate within the bearing 194 without the rod moving longitudinally relative to either the bearing or the rail 162. A knob or rotation means 186 may be connected to the second end 182 of the rod 178 and outside of the rail 162 to facilitate rotation of the rod by the practitioner.

The bracket 250 is made of steel, plastic or aluminum and includes a first portion 252 and a second portion 258, together having a generally L-shaped configuration. The second portion 258 includes a plurality of openings 260 extending entirely therethrough that are used to secure the rotating member 270 to the second portion using fasteners or the like. The first portion 252 includes a top surface 254 and a bottom surface 256. The bottom surface 256 is secured to a rotating member 290 that is subsequently secured to the distal end 118 of the arm 110 on the carriage assembly 60, as will be hereafter discussed.

The sleeve assembly 210 to be attached to the second rail assembly 160 includes a housing 212 and a sleeve 218 secured within the housing 212. The housing 212 has a proximal end 214 and a distal end 216, is generally square or rectangular, and is made of steel or aluminum. The sleeve 218 extends generally transverse to the housing 212 and is configured to receive an endoscope 12 of a predetermined size. The sleeve 218 is secured to the housing 212 via a bearing or the like (not shown) such that the sleeve is capable of rotating about its central axis 220 relative to the housing in the manner indicated by “A” in FIG. 7A.

A third locking mechanism 224 is secured to the distal end 216 of the housing 212 (FIG. 7A). Similar to the first 90 and second 132 locking mechanisms, the third locking mechanism 224 includes a clamp portion 226 having an opening 230 and a lever 228 that extends from the clamp portion and in a direction away from the housing 212. This configuration creates an L-shaped lever 228. The opening 230 includes internal threads 232 that are configured to mate with the external threads 184 on the rod 178 in the rail 162. The threads 184, 232 may be square, helical, or the like. The opening 230 is adapted to expand when the lever 228 is in a first, open condition, and to contract when the lever is in a second, locked condition. Thus, the third locking mechanism 224 is capable of providing an inwardly directed force in the closed condition. As will be hereafter described, the lever 228 operates to selectively engage opening 230 of the clamp 226 with the rod 178 disposed within the passage 172 of the rail 162.

The sleeve assembly 210 is mounted to the rod 178 by sliding the rod through the opening 230 in the third locking mechanism 224 when the lever 228 is in the open condition and, thus, the opening is in the expanded condition. This allows the sleeve assembly 210 to move relative to both the rod 178 and the rail 162 on which the rod is rotatably mounted. When the sleeve assembly 210 is mounted on the rod 178, the sleeve 218 is positioned substantially parallel to the passage 172 within the rail 162 in the second rail assembly 160. The third locking mechanism 224 is sized and configured such that, once the sleeve assembly 210 is mounted to the rod 178, the clamp portion 226 of the third locking mechanism fits within the passage 172 of the rail 162, and the lever 228 is positioned between the distal end 216 of the housing 212 and the front surface 188 of the rail 162 and extends over the top surface 164 of the rail 162.

Due to the engagement between the opening 230 in the third locking mechanism 224 and the rod 178, the sleeve assembly 210 is capable of moving back and forth relative to the rail 162 in the manner indicated by “G” in FIG. 7B. The range of travel of the sleeve assembly 210 along the rail 162 is limited to the distance between the end plate 174 at the proximal end 168 of the rail 162, the end plate 176 at the distal end 170 of the rail 162. In particular, the sleeve assembly 210 may move toward the distal end 170 of the rail 162 until the third locking mechanism 224 abuts the end plate 176, and toward the proximal end 168 of the rail until the third locking mechanism 224 abuts the end plate 176. Movement of the sleeve assembly 210 is accomplished by applying a force to the sleeve assembly substantially parallel to the rod 178 such that the static friction between the opening 230 in the third locking mechanism 224 and the periphery of the rod is overcome to allow the sleeve assembly to slide relative to the rod. This construction allows for quick, substantial movement of the sleeve assembly 210 along the rod 178 to a desired location.

The lever 228 is actuatable to selectively secure the sleeve assembly 210 to the rod 178. In particular, the lever 228 operates to selectively engage the threads 232 on the opening 230 of the clamp portion 226 of the third locking mechanism 224 with the threads 184 on the rod 178 by selectively expanding and contracting the opening in the clamp relative to the rod. When the lever 228 is in a first condition, indicated by solid lines in FIG. 7B, the clamp 226 is not fully engaged with the rod 178 and, thus, there exists only static friction between the clamp and the rod. In this condition, the sleeve assembly 210 can be moved along the rod 178 by applying force, as noted above, to overcome the static friction and make substantial movements of the sleeve assembly relative to the rod.

The lever 228 can be moved from the first condition to a second condition by rotating the lever, indicated at “R7”, relative to the clamp portion 226. When the lever 228 is in the second condition, indicated by dashed lines in FIG. 7B, the threads 232 on the opening 230 of the clamp 226 engage the threads 184 on the rod 178 and apply a force to the periphery of the rod sufficient to rigidly couple the rod to the clamp 226 and, thus, to the sleeve assembly 210. In this condition, the sleeve assembly 210 cannot be moved along the rod 178 by applying force due to the thread engagement and clamping force applied by the clamp 226 to the rod. Rather, the sleeve assembly 210 is moved by turning the rod 178 about its axis 187 via the knob 186, as indicated by “R5” in FIG. 7A. When the rod 178 is rotated, the threaded engagement between the threads 184 on the rod and the threads 232 on the opening 230 of the clamp 226 causes the sleeve assembly 210 to move relative to the fixed rod. This relative movement of the sleeve assembly 210 along the rod 178 is in small increments, or fine.

The indicia 190 on the front surface 188 of the rail 162 bear incremental measurements, e.g., inches, millimeters, etc., reflect the amount of longitudinal travel of the sleeve assembly 210 along the rod 178. Rotation of the knob 186 in a clockwise direction results in the sleeve assembly 210 moving toward the distal end 170 of the rail 162, whereas rotation of the knob in a counterclockwise direction results in the sleeve assembly moving toward the proximal end 168 of the rail. The relationship between the amount of rotation of the knob 186 and the correlative longitudinal travel distance of the sleeve assembly 210 along the rod 178 is based on the fineness of the threaded engagement between the rod and the opening 230 in the clamp 226. For example, rotation of the knob 186 in the clockwise direction 45° may cause the sleeve assembly 210 to move 3 mm towards the distal end 170 of the rail 162. However, if more threads per inch are present on the rod 178 and the opening 230 in the clamp 226, the same 3 mm movement of the sleeve assembly 210 may require 180° of clockwise rotation of the knob 186. This threaded engagement, therefore, determines how sensitive the sleeve assembly 210 is to rotation of the knob 186.

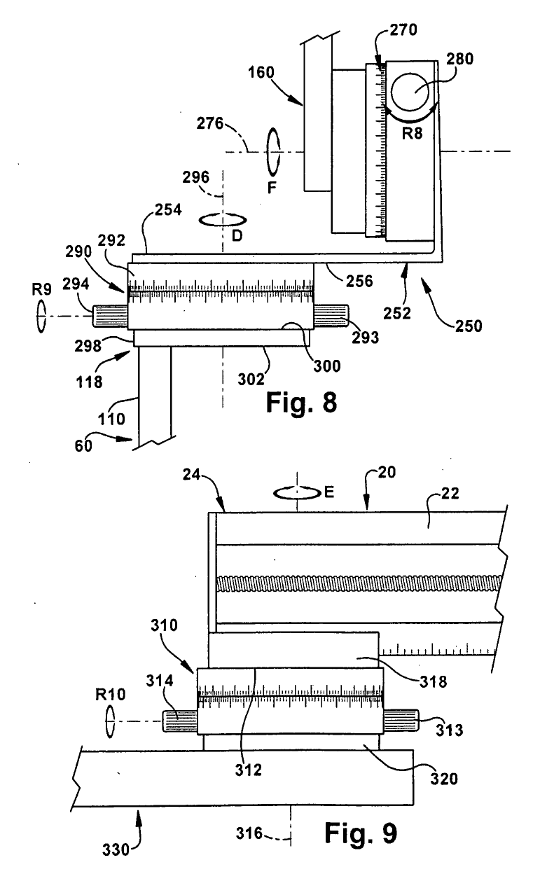

As shown in FIGS. 7B and 8, the endoscope 12 can be rotated with the sleeve 218 about the axis 220 in the manner A to provide further articulation of the present invention. The sleeve 218, and therefore the endoscope 12, can be rotated relative to the housing 212 by rotating a knob 222, indicated at “R6” connected to the sleeve. As with other aforementioned planes of rotation, indicia (not shown) across the face of the sleeve 218 are used to monitor the amount of rotation of the sleeve 218.

The correlation between the amount of rotation of the knob 222 and the amount of rotation of the sleeve 218 is based on the mechanical coupling between the knob and the sleeve. This coupling may comprise a pair of fine-toothed gears oriented orthogonal to one another, each gear having teeth adapted to translate rotation in one plane to rotation in a second, orthogonal plane (not shown). In such a case, the precision of the movement would be based on the amount of tooth engagement between the two gears. Those skilled in the art will appreciate that alternative means may be used to mechanically translate rotation of the knob 222 to rotation of the sleeve 218.

The bracket 250 and the second rail assembly 160 may also be rotated relative to one another via a rotating member 270. The rotating member 270 bears indicia 272 and is secured between the second portion 258 of the bracket 250 and the rear surface of the rail 178 of the second rail assembly 160. By coupling the rotating member 270 to the rail 162, the rail 162 and, thus, the second rail assembly 160 and sleeve assembly 210, can rotate about a central axis 276 of the rotating member 270 relative to the second portion 258 of the bracket 250 in the manner depicted by “F” (FIGS. 7B and 8).

A plate 282 having a recess 284 for receiving the rotating member 270 couples the rotating member to the bracket 250 and the second rail assembly 160. The recess 284 is sized such that only a portion of the rotating member 270 fits into the recess. A set screw 278 and a knob 280 are threadably engaged to the plate 282. The set screw 278 functions similar to the first, second, and third locking mechanisms 90, 132, 224 in that the set screw selectively prohibits rotation of the rotating member 270 and, thus, relative movement between the rail 178 and the bracket 250. In particular, the set screw 278 has a first, open condition disengaged from the rotating member 270, and a second, closed condition engaged with the rotating member 270 to thereby prohibit rotation of the rotating member.

When the set screw 278 is in the first condition, the application of force to the second rail assembly 160 creates a moment about the axis 276 and causes the second rail assembly to rotate in the manner F. When the set screw 278 is in the second condition, the set screw engages the rotating member 270 to prevent the second rail assembly 160 from rotating about the axis 276 under the application of force to the second rail assembly. The rotation of the second rail assembly 160 is instead accomplished by turning the knob 280. The knob 280 is mechanically coupled to the rotating member 270 such that rotation of the knob causes the rotating member to rotate about the axis 276 to articulate the second rail assembly 160 and sleeve assembly 210 relative to the bracket 250.

The indicia 272 around the periphery of the rotating member 270 bear incremental measurements, e.g., inches, millimeters, etc., and reflect the amount of rotation of the rail 162 relative to the bracket 250. Rotation of the knob 280 in a clockwise direction results in the rail 162 rotating in a counterclockwise direction, whereas rotation of the knob in a counterclockwise direction results in the rail rotating in a clockwise direction. The relationship between the amount of rotation of the knob 280 and the correlative rotation of the rail 162 is based on the fineness of the threaded engagement between the knob and the rotating member 270. For example, rotation of the knob 280 in the clockwise direction 45° may cause the rail 162 to rotate 10° in the counterclockwise direction. However, if more threads per inch are present on the knob 280 and rotating member 270, the same 10° movement of the rail 162 may require 180° of clockwise rotation of the knob. This threaded engagement, therefore, determines how sensitive the rail 162 is to rotation of the knob 280.

As shown in FIGS. 8-9, a mounting plate 298 and rotating member 290 couple the second rail assembly 160 and the sleeve assembly 210 to the carriage assembly 60. The mounting plate 298 includes a top surface 300 and a bottom surface 302. The bottom surface 302 is secured by fasteners to the distal end 118 of the arm 110 on the carriage assembly 60 such that the mounting plate is orthogonal to the arm. The rotating member 290 is then secured to the top surface 300 of the plate 298 and the bottom surface 256 of the first portion 252 of the bracket 250. The rotating member 290 allows the second rail assembly 160 and the sleeve assembly 210 to rotate relative to the carriage assembly 60 and the first rail assembly 20.

A set screw 293 and a knob 294 are threadably engaged to the rotating member 290. The set screw 293 functions similar to the first, second, and third locking mechanisms 90, 132, 224 in that the set screw selectively prohibits rotation of the rotating member 290 and, thus, relative movement between the bracket 250 and carriage assembly 60. In particular, the set screw 293 has a first, open condition disengaged from the rotating member 290, and a second, closed condition engaged with the rotating member to thereby prohibit rotation of the rotating member.

When the set screw 293 is in the first condition, the application of force to the bracket 250 creates a moment about the axis 296 and causes the bracket to rotate in the manner indicated at “D”. When the set screw 293 is in the second condition, the set screw engages the rotating member 290 to prevent the bracket 250 from rotating about the axis 296 under the application of force applied to the bracket. The rotation of the bracket 250 is instead accomplished by turning the knob 294. The knob 294 is mechanically coupled to the rotating member 290 such that rotation of the knob causes the rotating member to rotate about the axis 296 to articulate the bracket 250 and, thus, the second rail assembly 160 and sleeve assembly 210, relative to the carriage assembly 60.

Rotation of the knob 294 in the direction indicated by “R9” causes the rotating member 290 to rotate about the central axis 296 in the manner D. This causes the bracket 250, and therefore the second rail assembly 160 and sleeve assembly 210, to rotate relative to the carriage assembly 60 about the axis 296. Indicia 292 around the periphery of the outside of the rotating member 290 bear incremental measurements, e.g., inches, millimeters, etc., and reflect the amount of rotation of the second rail assembly 160 relative to the carriage assembly 60. Rotation of the knob 294 in a clockwise direction results in the bracket 250 rotating in a counterclockwise direction, whereas rotation of the knob in a counterclockwise direction results in the bracket rotating in a clockwise direction. The relationship between the amount of rotation of the knob 294 and the correlative rotation of the bracket 250 is based on the fineness of the threaded engagement between the knob and the rotating member 290. For example, rotation of the knob 294 in the clockwise direction 45° may cause the bracket 250 to rotate 10° in the counterclockwise direction. However, if more threads per inch are present on the knob 294 and rotating member 290, the same 10° movement of the bracket 250 may require 180° of clockwise rotation of the knob. This threaded engagement, therefore, determines how sensitive the bracket 250 is to rotation of the knob 294.

As illustrated in FIGS. 1 and 9, two mounting brackets 318, 320 and a rotating member 310 rotatably couple the entire guidance system 10 to a cart or table 330. One mounting bracket 318 secures the proximal end 24 of the rail 22 of the first rail assembly 20 to the rotating member 310. The other bracket 320 secures the rotating member 310 to the table 330. This enables the entire guidance system 10 to be rotatable relative to the table 330. The system 10 is rotatable by rotating the first rail assembly 20, to which the second rail assembly 160, carriage assembly 60, and sleeve assembly 210 are mounted, relative to the rotating member 310.

A set screw 313 and a knob 314 are threadably engaged to the rotating member 310. The set screw 313 functions similar to the first, second, third locking mechanisms 90, 132, 224 in that the set screw selectively prohibits rotation of the rotating member 310 and, thus, relative movement between the first rail assembly 20 and the table 330. In particular, the set screw 313 has a first, open condition disengaged from the rotating member 310, and a second, closed condition engaged with the rotating member 310 to thereby prohibit rotation of the rotating member.

When the set screw 313 is in the first condition, the application of force to the first rail assembly 20 creates a moment about the axis 316 and causes the first rail assembly to rotate in the manner indicated at “E”. When the set screw 313 is in the second condition, the set screw engages the rotating member 310 to prevent the first rail assembly 20 from rotating about the axis 316 under the application of force applied to the first rail assembly 20. The rotation of the first rail assembly 20 is instead accomplished by turning the knob 314. The knob 314 is mechanically coupled to the rotating member 310 such that rotation of the knob causes the rotating member to rotate about the axis 316 to articulate the first rail assembly 20, and thus the second rail assembly 160, sleeve assembly 210, and carriage assembly 60, relative to the table 330.

Rotation of a knob 314 in the direction indicated by “R10” causes the rotating member 310 to rotate about the central axis 316 in the manner E. This causes the first rail assembly 20 to rotate about the axis 316. Indicia 312 around the periphery of the rotating member 310 bear incremental measurements, e.g., inches, millimeters, etc., and reflect the amount of rotation of the second rail assembly 160 relative to the carriage assembly 60. Rotation of the knob 314 in a clockwise direction results in the first rail assembly 20 rotating in a counterclockwise direction, whereas rotation of the knob in a counterclockwise direction results in the first rail assembly rotating in a clockwise direction. The relationship between the amount of rotation of the knob 314 and the correlative rotation of the first rail assembly 20 is based on the fineness of the threaded engagement between the knob and the rotating member 310. For example, rotation of the knob 314 in the clockwise direction 45° may cause the first rail assembly 20 to rotate 10° in the counterclockwise direction. However, if more threads per inch are present on the knob 314 and rotating member 310, the same 10° movement of the first rail assembly 20 may require 180° of clockwise rotation of the knob. This threaded engagement, therefore, determines how sensitive the first rail assembly 20 is to rotation of the knob 314.

FIG. 10 depicts the guidance system 10 in the fully assembly condition. In this condition, the endoscope 12 secured within the sleeve 218 is capable of articulation and placement in several degrees of freedom due to the configuration of the system 10. Since the first rail assembly 20, second rail assembly 160, carriage assembly 60, and sleeve assembly 210 are all interconnected, articulation of one results in the movement of the others, thereby allowing for very precise movement of the endoscope 12. As shown, the endoscope 12 is capable of movement in seven degrees of freedom. Movement in the manner A is possible by rotating the knob 222 on the sleeve assembly 210 to rotate the sleeve 218 about its axis 220 in the manner A.

If the first locking mechanism 90 is in the first, unlocked condition, the application of force to the carriage assembly 60 results in substantial movement of the carriage assembly in the manner B. If the first locking mechanism 90 is in the second, locked condition, rotation of the knob 46 on the first rail assembly 20 results in precise movement of the carriage assembly in the manner B along the rod 36 in the first rail assembly 20, which likewise causes the second rail assembly 160 and sleeve assembly 210 to move.

If the second locking mechanism 132 is in the first, unlocked condition, the application of force to the carriage 100 results in substantial movement of the carriage in the manner C. If the second locking mechanism 132 is in the second, locked condition, rotation of the knob 128 on the carriage assembly 60 results in precise movement of the carriage in the manner C along the rod 120 in the carriage assembly 60, which likewise causes the second rail assembly 160 and sleeve assembly 210 to move.

If the third locking mechanism 224 is in the first, unlocked condition, the application of force to the sleeve assembly 210 results in substantial movement of the carriage in the manner G. If the third locking mechanism 224 is in the second, locked condition, rotation of the knob 186 on the second rail assembly 160 results in precise movement of the sleeve assembly 210 in the manner G along the rod 178 in the second rail assembly 160.

The sleeve assembly 210 and second rail assembly 160 can be rotated about the axis 296 in the manner D by rotating the knob 294 on the rotating member 290. The sleeve assembly 210 and second rail assembly 160 can also be rotated about the axis 276 in the manner F by rotating the knob 280 on the rotating member 270. The entire guidance system 10 can be rotated relative to the table 330 or platform to which it is secured by rotating the knob 314 on the rotating member 310 to cause the system 10 to rotate in the manner E.

Due to the capability of each range of motion to be undertaken with precision, practitioners can rely on repeatable and accurate endoscopic placement within the patient 14. The stability of the guidance system 10 further allows a single surgeon to manipulate and articulate the endoscope 12 to the desired location while maintaining the ability to feed endoscopic instrumentation down the lumen of the endoscope to perform the requisite diagnostic or therapeutic procedure.

The guidance system 10 is further capable of being used for telesurgical endoscopic guidance for performing such procedures as treating obstructing kidney stones in remote locations without ready access to endoscopic expertise. The guidance system can also be used with all other forms of endoscope, and for treatments or diagnostics performed with these endoscopes. In such a situation, or generally whenever a surgeon at one location wishes to guide another surgeon or practitioner in using the guidance system 10 via radio, satellite communication or the like, the guidance system 10 can be color-coded such that one surgeon can efficiently and effectively communicate desired manipulation commands to the other surgeon.

For example, each degree of freedom in the guidance system 10 can be given a color—e.g. the knob 46 used to move the carriage assembly 60 in the manner B can be painted yellow, and the knob 222 used to rotate the sleeve 218 within the sleeve assembly 210 in the manner A can be painted green. This configuration would allow an endoscopist outside of the operating room to watch a live video feed of the endoscope 12 within the patient 14 and direct a bedside assistant to, for example, turn the yellow dial clockwise until the carriage moves 20 mm toward the proximal end 24 of the first rail assembly 20, as indicated by the indicia 48 on the front of the rail 22. Therefore, the present invention provides a simple to use, hand-guided, motorless system to guide an endoscope with high precision, either directly or in a telesurgical application.

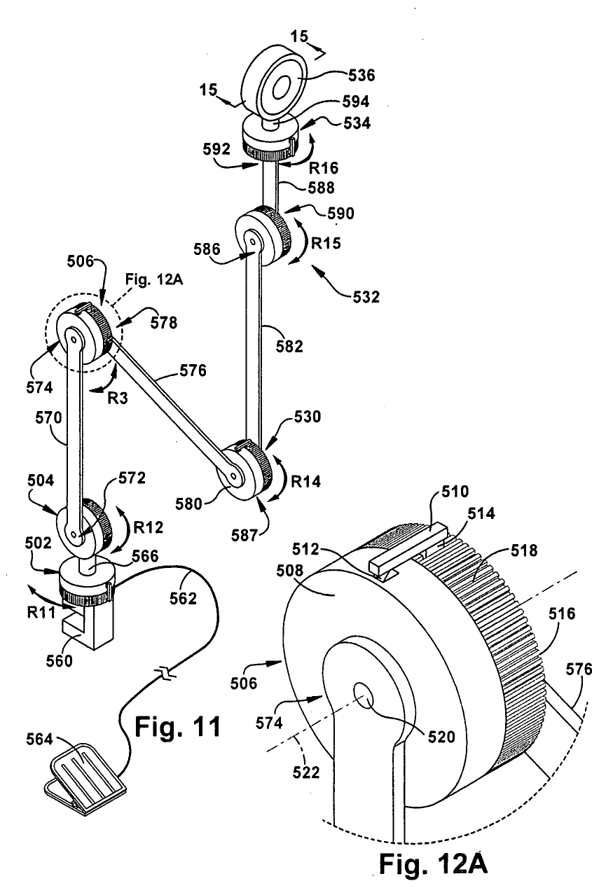

A guidance system 500 in accordance with a second embodiment of the present invention is depicted schematically in FIG. 11. The system 500 includes a series of support rods 566, 570, 576, 582, 588, 594 rotatably mounted relative to one another by a series of rotating stages 502, 504, 506, 530, 532, 534 and a sheath 536. The system 500 is mounted to a table (not shown) via a clamp 560 for stability, and is used for movement and articulation of an endoscope disposed within the sheath 536. The system 500 is capable of movement in several degrees of freedom, indicated at “R11-R16”, in order to facilitate accurate placement of the endoscope. Similar to the system 10, the system 500 can be constructed of metal, plastic, or any other light-weight durable material. Although six stages and six support rods are illustrated and described, those skilled in the art will appreciate that any number of stages and supports may be utilized. Furthermore, it shall be understood that each stage may be oriented in a horizontal plane, a vertical plane, or a plane angled relative thereto to provide the desired degrees of freedom of movement of the endoscope.

The device 500 further includes a cable 562 that will supply an electromagnetic force to the device 500 from a power source (not shown). A foot pedal 564 couples the cable 562 to the power source. The endoscopist activates the foot pedal 564 to selectively provide the electromagnetic signal to the cable 562 and, thus, the device 500.

As shown in FIG. 11, the first rod 566 of the device 500 has a proximal end 567 secured to the first stage 502 and a distal end 568 secured to the second stage 504. The second rod 570 has a proximal end 572 secured to the second stage 504 and a distal end 574 secured to the third stage 506. The third rod 576 has a proximal end 578 secured to the third stage 506 and a distal end 580 second to the fourth stage 530. The fourth rod 582 has a proximal end 584 secured to the fourth stage 530 and a distal end 586 secured to the fifth stage 532. The fifth rod 588 has a proximal end 590 secured to the fifth stage 532 and a distal end 592 secured to the sixth stage 534. The sixth rod 594 has a proximal end 596 secured to the sixth stage 534 and a distal end 598 secured to the sheath 536. Each support rod is generally tubular and constructed of a material capable of supporting the weight of the device 500 without plastic deformation. Each of the rods has a series of wires disposed therein (not shown) that are used to electrically couple each stage to the next, as will be hereafter described.

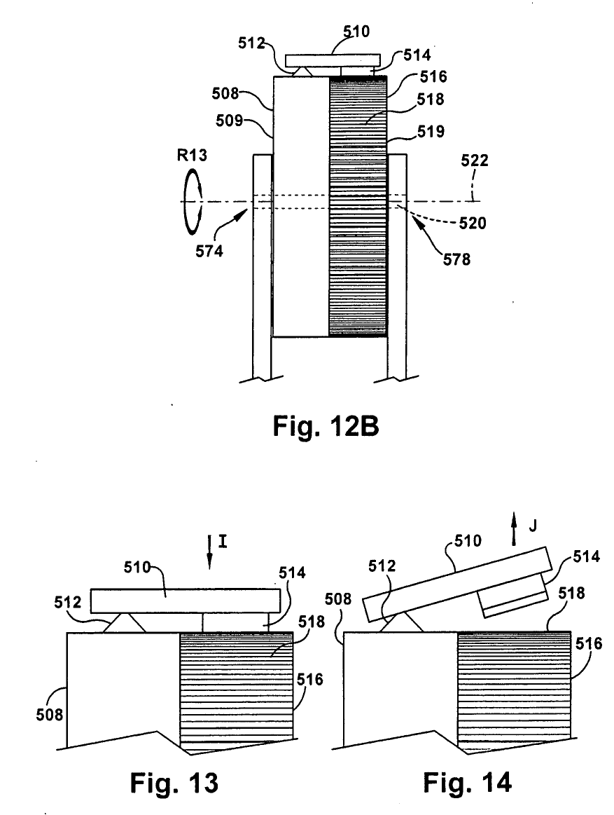

The details of the third stage 506 are illustrated in FIGS. 12A-14. It will be understood, however, that the first stage 502, second stage 504, fourth stage 530, fifth stage 532, and sixth stage 534 are substantially identical to the third stage 506. The third stage 506 includes a first wheel 508 and second wheel 516. The distal end 574 of the second rod 570 is secured to an outer surface 509 of the first wheel 508 via a pin 520 such that the first wheel is incapable of rotating relative to the distal end 574 of the second rod 570. The pin 520 also secures the proximal end 578 of the third rod 576 to an outer surface 519 of the second wheel 516 such that the second wheel is incapable of rotating relative to the proximal end of the third rod. The pin 520 also interconnects the first wheel 508 to the second wheel 516 such that the second wheel is capable of rotating about a central axis 522 of the pin 520 relative to the first wheel. This may be accomplished by use of a bearing (not shown) coupling the second wheel 516 to the pin 520 and the third rod 576.

The first wheel 508 is generally circular and has secured to its periphery an arm 510 that is actuatable to selectively prohibit rotation of the second wheel 516 relative to the first wheel. The arm 510 extends substantially parallel to the axis 522 of the pin 520 and may be constructed of magnetic material. The arm 510 is pivotally secured to the periphery of the first wheel 508 by a hinge 512. The hinge 512 may exhibit a triangular shape or otherwise be adapted to provide a location about which the arm 510 may pivot relative to the first wheel 508. A projection 514 extends away from the arm 510 in a downward direction towards the central axis 522 of the pin 520.

The first wheel of every stage will have secured thereto or disposed therein a solenoid or other means (not shown) for transmitting an electromagnetic signal supplied by the cable 562. The wires disposed within each rod electrically connect each stage to each subsequently numbered stage and ultimately each stage to the cable 562. For example, the wires disposed within the third rod 576 will electrically connect the solenoid in the first wheel 508 of the third stage 506 to the solenoid in the first wheel of the fourth stage 530, the wires disposed within the fourth rod 582 will electrically connect the solenoid in the first wheel of the fourth stage to the solenoid in the first wheel of the fifth stage 532, and so on (see FIG. 11). This electrical pathway is electrically connected to the cable 562 and ultimately to a power source, such as a wall outlet. Furthermore, the sheath 536 may include similar connectivity means such that the sheath is electrically connected to the cable 562.

The second wheel 516 on the third stage 506 is generally circular and has disposed on its periphery a toothed or cogged surface 518 extending substantially parallel to the central axis 522 of the pin 520. The toothed surface 518 is configured to mate with the projection 514 on the arm 510 of the first wheel 508. Due to the electrical configuration of the device 500, an electromagnetic signal can be selectively applied to the first wheel 508 of each stage to selectively engage and disengage the projection 514 on the arm 510 with the toothed surface 518 on the second wheel 516.

The foot pedal 564 (FIG. 11) can be actuated to selectively supply an electromagnetic signal to the cable 562, and thus the device 500, from the power source. The pedal 564 may include a switch that is in the normally closed condition such that, when the pedal 564 is not activated, i.e., engaged, the switch remains closed and the device 500 is supplied with an electromagnetic signal. In this construction, when the foot pedal 564 is not activated the electromagnetic signal generated by the power source travels through the pedal 564, the cable 562, and eventually to each stage. This causes the solenoid in the first wheel 508 to energize. The energized solenoid imparts an electromagnetic force upon the arm 510 such that the arm rotates about the hinge 512 and moves in a downward direction, indicated at “1”, to engage the toothed surface 518 on the second wheel 516. This prevents the second wheel 516 from rotating relative to the first wheel 508, thereby locking the third stage 506. Since each vertical and horizontal stage exhibits this locking configuration, the activation of the electromagnetic signal locks all of the stages such that the device 500 is locked in its current position.

When the pedal 564 is activated, the internal switch on the pedal opens, thereby breaking the circuit. This de-energizes solenoid in the third stage 504 and causes the arm 510 and, thus, the projection 514, to pivot about the hinge 512 in an upward direction, indicated at “J” in FIG. 14 away from the toothed surface 518 of the second wheel 516. This movement disengages the projection 514 from the toothed surface 518. This activation likewise causes the remaining solenoids in the device 500 to de-energize, releasing each projection from each respective toothed surface on every stage.

With the second wheel 516 on the third stage 504 unencumbered, the second wheel is capable of rotating about the central axis 522 of the pin 520 relative to the first wheel 508. The second wheel 516 can be rotated relative to the first wheel 508 by applying a force to the third rod 576 because the third rod is rigidly secured to the outer surface 519 of the second wheel 516. This is likewise true of the fourth rod 582 relative to the fourth stage 530, the fifth rod 588 relative to the fifth stage 590, and so on. The activation of the pedal 564 therefore places the entire device 500 in an unlocked and freely moveable state.

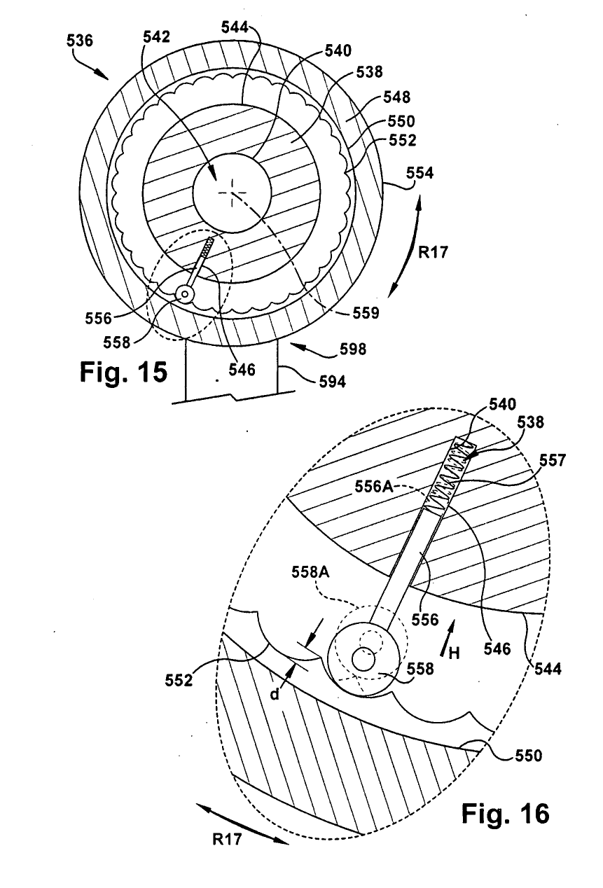

Articulation of the sheath 536 that holds the endoscope is illustrated in FIGS. 15-16. The sheath 536 includes a first cylinder 538 concentrically disposed within a second cylinder 548 about a common axis 559. The first cylinder 538 is tubular and includes an inner surface 540 and an outer surface 544. The inner surface 540 defines an opening 542 for receiving an endoscope. An aperture 546 extends from the outer surface 544 towards the inner surface 540.

The second cylinder 548 is tubular and includes an inner surface 550 and an outer surface 554. The second cylinder 548 is secured to the distal end 598 of the sixth rod 594 to prevent the second cylinder from rotating about the axis 559 relative to the sixth rod. The inner surface 550 of the second cylinder 548 includes a toothed surface configuration 552 adapted to releaseably engage a wheel 558 disposed between the outer surface 544 of the first cylinder 538 and the inner surface of the second cylinder. The wheel 558 is secured to the first cylinder 538 by a lever 556 and a spring 557 that are positioned substantially within the aperture 548 of the first cylinder 538. The lever 556 may have a telescoping construction and be made of magnetic material.

The engagement between the wheel 558 and the toothed surface 552 allows the endoscope to be rotated 360° with the first cylinder 538, indicated by “R17”, relative to the fixed second cylinder 548. In particular, when the first cylinder 538 is rotated by the practitioner the wheel 558 travels along the toothed surface 552 of the second cylinder 548. As the wheel 558 approaches the onset of the next tooth around the periphery of the inner surface 550 of the second cylinder 548, the wheel is forced in the direction “H” into a retracted position towards the aperture 546 in the first cylinder 538, as indicated by phantom lines 556a, 558a in FIG. 16. The amount of force required to rotate the first cylinder 538 is dependent upon the depth of the toothed surface 552, indicated at “d”, as well as the spring constant of the spring 557. This retraction is possible due to compression of the spring 557 or telescoping of the lever 556 or both.

When the wheel 558 passes a tooth, the spring force causes the spring 557 to expand, thereby forcing the wheel away from the aperture 546 into the extended position closer to the inner surface 552 of the second cylinder 548. This reciprocating movement of the wheel 558 is repeated until the first cylinder 538 and, thus, the endoscope is rotated into the correct position relative to the second cylinder 548. The engagement and spring 557 ensure that at least some rotational force must be applied to the first cylinder 538 in order to overcome the spring force of the spring 557 and cause the endoscope within the first cylinder to change position relative to the second cylinder 548.

The sheath 536 may further include a locking mechanism for selectively locking the lever 556. In such a construction, the lever 556 is electrically coupled to a solenoid or other means capable of electrical connectivity disposed in or secured on the sheath 536. The solenoid is also electrically coupled to the sixth stage 534 by wires disposed in the sixth rod 594 (not shown). The solenoid may be configured such that, when an electromagnetic signal is supplied, an electromagnetic force is applied to the lever 556 to cause movement of the lever in a direction away from the first cylinder 538 and towards the second cylinder 548. This forces the wheel 558 against the toothed surface 552 on the inner surface 550 of the second cylinder 548 and thereby creates a resistance to rotation of the second cylinder relative to the first cylinder 538. This resistance to rotation is greater than the resistance produced by the height do of the toothed surface 552 and the spring constant of the spring 557. Although the stages and the sheath 536 are illustrated as exhibiting different locking mechanisms, either or both of the mechanisms may be utilized with each stage and the sheath.

When the device 500 is in the unlocked and freely movable state, the device can move freely with the endoscope guided directly by the skilled endoscopist. When the endoscopist guides the endoscope into the proper location to perform a procedure, the endoscopist can lock the guidance system 500 into that precise location by disengaging the foot pedal 564. This sends the electromagnetic signal throughout the device 500, thereby energizing the solenoids to lock all of the stages, as well as the sheath 536, in place. This frees the endoscopist to turn away from the patient to gather or exchange instruments and to utilize the lumen of the endoscope to perform therapeutic or diagnostic procedures. With both hands free, the endoscopist can use the intralumenal endoscopic tools to perform any procedure directly instead of being forced to hold the endoscope and have a required second endoscopist perform any procedure while the endoscope is held by the first endoscopist. This system 500 thereby effectively doubles the efficiency of endoscopic procedures by allowing two endoscopists to perform endoscopy individually instead of together.

From the above description of the invention, those skilled in the art will perceive improvements, changes and modifications. Such improvements, changes and modifications within the skill of the art are intended to be covered by the appended claims.

Having described the invention, the following is claimed:

1. An endoscopic guidance system comprising
an endoscope;
a first rail assembly having a rail member and a first rod secured thereto, said first rod being rotatable relative to said rail member by rotation means; and
a sleeve assembly coupled to the first rod, wherein the endoscope is releasably engageable with the sleeve assembly;
wherein rotation of the first rod causes the sleeve assembly to move along the first rod to guide the endoscope to a desired position along a first degree of freedom.

2. The apparatus of claim 1, wherein the sleeve assembly comprises a housing and a sleeve rotatably mounted therein, the endoscope being secured in the sleeve and rotatable within the sleeve to a desired position along a second degree of freedom.

3. The apparatus of claim 2 further comprising a bracket having a first portion and a second portion, the sleeve assembly being secured to the second portion of the bracket by a first rotating member, wherein the rotating member rotates relative to the bracket to move the endoscope to a desired position along a third degree of freedom.

4. The apparatus of claim 3, wherein a second rotating member is secured to the first portion of the bracket, the bracket being rotatable relative to the second rotating member to move the endoscope to a desired position along a fourth degree of freedom.

5. The apparatus of claim 4 further comprising a carriage assembly, the carriage assembly comprising:
a base member and a second rod secured thereto, the second rod being rotatable relative to the base;
a carriage coupled to the second rod, the carriage having an arm extending substantially parallel to the second rod, the second rotating member being secured to a distal end of the arm;
wherein rotation of the second rod causes the sleeve assembly to move along the second rod to guide the endoscope to a desired position along a fifth degree of freedom.

6. The apparatus of claim 5 further comprising a second rail assembly, the second rail assembly comprising:
a rail member and a third rod secured thereto, said third rod being rotatable relative to said rail member by rotation means;
wherein the base of the carriage assembly is coupled to the third rod;
wherein rotation of the third rod causes the carriage assembly to move along the third rod to guide the endoscope to a desired position along a sixth degree of freedom.

7. The apparatus of claim 6 further comprising a third rotating member, the second rail assembly being secured to the third rotating member and rotatable relative to the third rotating member to move the endoscope to a desired position along a seventh degree of freedom.

8. A method for manually guiding an endoscope, the method comprising the steps of:
providing an endoscope;
providing a first rail assembly having a rail member and a first rod secured thereto, the first rod being rotatable relative to the rail member by rotation means;
providing a sleeve assembly;
coupling the sleeve assembly to the first rod;
securing the endoscope within the sleeve assembly;
rotating the first rod to cause the sleeve assembly to move along the first rod to guide the endoscope to a desired position along a first degree of freedom.
